(19) United States
(12) Patent Application Publication
(10) Pub. No.: US 2011/0132375 A1
(43) Pub. Date: Jun. 9, 2011

(54) System and Method for Forming a Custom-Molded Medical Mask

(75) Inventor: W. Keith Thornton, Dallas, TX (US)

(73) Assignee: AirWay Technologies, LLC, Carrollton, TX (US)

(21) Appl. No.: 12/956,242

(22) Filed: Nov. 30, 2010

Related U.S. Application Data
(60) Provisional application No. 61/267,500, filed on Dec. 8, 2009.

(51) Int. Cl. A61M 16/06; B29C 43/02

(52) U.S. Cl. 128/206.24; 264/319

(57) ABSTRACT

A custom medical mask includes a substantially rigid custom-fitted sealing ridge that has been shaped to correspond to a compressed shape of soft tissues of a user's face. The sealing ridge includes first, second, and third ridges. The first ridge corresponds to a first portion of the user's face extending from the user's nose to a first area proximate a lateral perimeter of the user's obicularis-oris muscle. The second ridge corresponds to a second portion of the user's face extending from the nose to a second area proximate a lateral perimeter of the obicularis-oris muscle. The third region corresponds to the lower extension of the obicularis-oris muscle extending from the first area to the second area. The sealing ridge is configured to form a custom-fit seal by conforming to the compressed shape of the user's soft tissues when the mask is pressed against the user's face.

### RELATED APPLICATIONS

This application claims the benefit under 35 U.S.C. §119(e) of U.S. Provisional Application No. 61/267,500 filed Dec. 8, 2009, entitled “System and Method for Forming a Custom-Molded Medical Mask”.

### TECHNICAL FIELD

This invention relates generally to masks for use in medical and other clinical applications, and more particularly to a system and method for forming a custom-molded medical mask.

### BACKGROUND

Many people experience breathing problems on a recurring basis, which often result in sleep disordered breathing (i.e., difficulty sleeping, snoring, or other more serious conditions such as obstructive sleep apnea). As technology advances, people with such breathing problems demand increased performance and comfort. Previous devices for improving a user's breathing have included face masks, nose masks, or nasal inserts that help deliver air to the particular user's nose at positive pressure. These devices help force open the particular user's breathing passage and thereby improve the user's breathing. However, previous devices have often provided an inadequate fit and thus have caused discomfort for users and failed to adequately prevent leakage.

### OVERVIEW

According to one embodiment, a method of forming a custom medical mask with a substantially rigid custom-fitted sealing ridge is disclosed. The custom-fitted sealing ridge is custom shaped to correspond to a compressed shape of soft tissues of a particular user's face. The method includes transitioning a mask blank to a substantially deformable state. The mask blank is positioned to cover the particular user's mouth and at least a portion of the particular user's nose. The mask blank is pressed against first, second, and third facial regions of the particular's users face. The first facial region extends from the particular user's nose to a first area proximate a lateral perimeter of the particular user's obicularis-oris muscle. The first area is located to the right of the particular user's mouth. The second facial region extends from the particular user's nose to a second area proximate a lateral perimeter of the particular user's obicularis-oris muscle. The second area is located to the left of the particular user's mouth. The third facial region extends from the first area to the second area, the third region corresponding to the lower extension of the particular user's obicularis-oris muscle. The substantially rigid custom-fitted sealing ridge conforms to the compressed shape of the particular user's soft tissues when the mask blank is pressed against the first, second, and third facial regions of the particular user's face.

According to another embodiment, a custom medical mask includes a substantially rigid custom-fitted sealing ridge that has been custom shaped to correspond to a compressed shape of soft tissues of a particular user's face. The substantially rigid custom-fitted sealing ridge includes first, second, and third ridges. The first ridge corresponds to a first portion of the particular user's face extending from the particular user's nose to a first area proximate a lateral perimeter of the particular user's obicularis-oris muscle. The first area is located to the right of the particular user's mouth. The second ridge corresponds to a second portion of the particular user's face extending from the particular user's nose to a second area proximate a lateral perimeter of the particular user's obicularis-oris muscle. The second area is located to the left of the particular user's mouth. The third ridge corresponds to a third portion of the particular user's face extending from the first area to the second area. The third portion corresponds to the lower extension of the particular user's obicularis-oris muscle. The substantially rigid custom-fitted sealing ridge is configured to form a custom-fit seal by conforming to the compressed shape of the particular user's soft tissues when the custom medical mask is pressed against the first, second, and third portions of the particular user's face. Certain embodiments may provide one or more technical advantages. Certain embodiments may provide a custom medical mask that conforms substantially optimally to a user's unique facial features. Certain embodiments may provide a custom medical mask that may couple to an oral appliance to provide an improved fit based on the precise custom orientation or positional relationship between a compressed shape of the user's unique facial structure and a custom-fitted sealing ridge of the mask. Various masks may have portions that are integrally formed using cost-efficient processing and that may have enhanced durability. Certain embodiments may provide a process for use in forming a custom medical mask that maintains a precise custom orientation or positional relationship through each step of the process to ensure that the custom medical mask has an optimal fit, customized for the user. Certain embodiments may provide improved fit, increased comfort, reduced leakage, and improved performance, whether for treating sleep disordered breathing, administering anesthesia or other medical gases, or any other suitable purpose for which the custom medical mask is used. Certain embodiments may provide all, some, or none of these advantages. Certain embodiments may provide one or more other technical advantages, one or more of which may be apparent to those skilled in the art from the figures, descriptions, and claims included herein.

### BRIEF DESCRIPTION OF THE DRAWINGS

For a more complete understanding of the present invention and certain of its advantages, reference is now made to the following description, taken in conjunction with the accompanying drawings, in which:

FIG. 1 illustrates a portion of an example system for improving a user's breathing including an example custom-fitted mask according to one embodiment;

FIG. 2 illustrates a portion of an example system for improving a user's breathing including an example custom-fitted mask according to an alternative embodiment;

FIG. 3A illustrates muscles of a particular user's face;

FIG. 3B illustrates other soft tissues of a particular user's face;

FIGS. 4A through 4D illustrate an example method of forming a custom-fitted mask according to one embodiment;

FIGS. 5A through 5D illustrate various cross-sections of an example custom-fitted mask at different stages of formation;

FIG. 6 illustrates an alternative mask custom-fitted to another particular user according to one embodiment; and

FIG. 7 illustrates a flowchart of an example method for forming a custom-fitted mask for a particular user according to one embodiment.

### DESCRIPTION OF EXAMPLE EMBODIMENTS

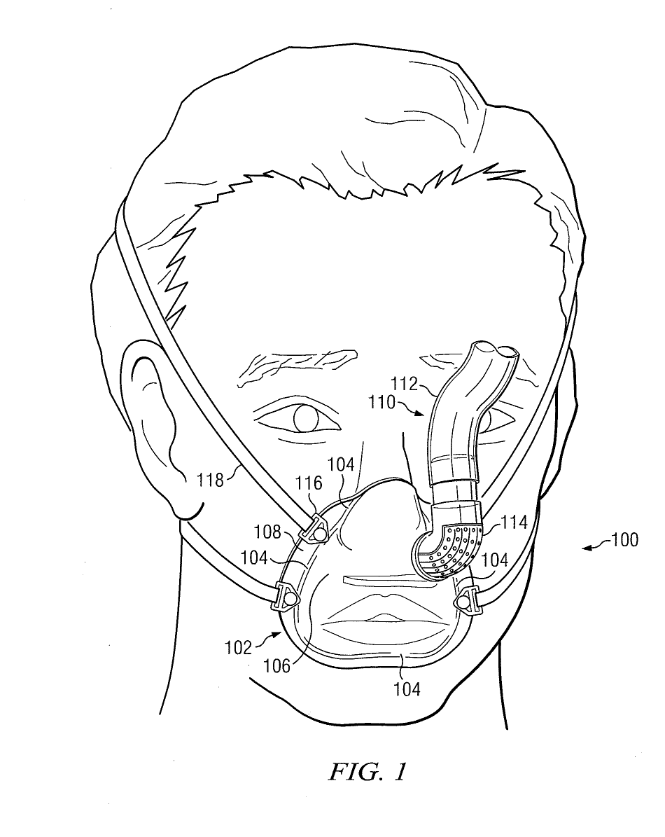

FIG. 1 illustrates a portion of an example system 100 for improving a user's breathing including an example custom-fitted mask 102 according to one embodiment. Mask 102 includes a substantially rigid, custom-fitted sealing ridge 104. In particular embodiments, sealing ridge 104 may define the perimeter of a sealed enclosure or chamber 106 capable of receiving gas when mask 104 is pressed against soft tissues of the particular user's face. In certain embodiments, chamber 106 106 may define a space within which air or another gas may be supplied to the user's nose at positive pressure to help open the user's breathing passage and thereby improve the user's breathing. The term “soft tissue” as used herein generally refers to any muscle, skin, connecting tissue, cartilage, tendons, ligaments, fascia, fibrous tissues, fat, etc. that may be compressed by an applied pressure and that connect, support, and/or surround other structures and organs of the body.

Sealing ridge 104 has been custom-shaped to correspond to a compressed shape of soft tissues of a particular user's face. The custom-shape of sealing ridge 104 may enable sealing ridge 104 to form a custom-sealed chamber 106 by pressing sealing ridge 104 against soft tissues of the user's face. In certain embodiments, sealing ridge 104 may provide improved fit, reduced leakage, increased comfort, and better performance and durability, whether in treating breathing problems such as sleep disordered breathing or for any other suitable purpose for which the custom-fitted mask 102 is used. The hard seal provided by pressing a substantially rigid, custom-fitted sealing ridge 104 against soft tissues of the user's face may be contrasted with alternative soft-seal masks that form airtight seals with a user's face by deforming a substantially flexible contact portion of the mask.

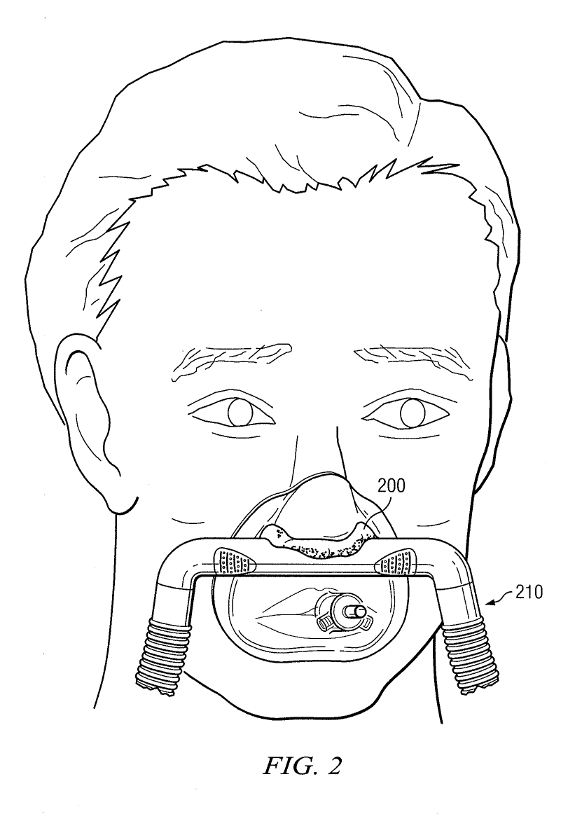

In particular embodiments, mask 102 may include one or more holes 105 that permit flow of gas into and out of mask 102. In certain embodiments, the hole or holes 105 may be used to couple mask 102 to an external system 110, such as a continuous positive air pressure (CPAP) system, a bi-level positive airway pressure (BiPAP) system, and/or alternative gas delivery systems. For example, system 100 may include one or more tubes 112 that snap onto, are force fitted onto, or otherwise couple to fittings 114. In an alternative embodiment, a sealant 200 may be used to form an air-tight coupling of mask 102 and an external system 210, as shown in FIG. 2. Various embodiments may include a mask having dual chambers, as shown in FIG. 1, and/or may include a receptacle or other structures that may be used to couple mask 102 to an oral appliance, as shown in FIG. 2. Although the masks 102 of FIGS. 1 and 2 illustrate dual-chamber and single-chamber masks, respectively, mask 102 may have any suitable number of chambers. Various dual-chamber and single-chamber masks are disclosed in U.S. Patent Application Nos. 2007/0006879 and 2008/0006274, each of which are herein incorporated by reference.

In certain embodiments, mask 102 may further include a flange 108 portion disposed exterior to and around sealing ridge 104 and internal sealed region 106. Flange 108 may or may not conform to particular features of the user's face. As shown in FIG. 1, for example, a perimeter of flange 108 curves slightly away from the user's face and is not be in direct contact with the user's face. In various embodiments, the flange 108 portion of mask 102 may include buckles or other devices 116 to couple mask 102 to one or more straps 118, which may help secure mask 102 to the user's face during use. Devices 116 may be coupled to mask 102 during or after formation of mask 102. Although straps 118 are described as an example, mask 102 may be secured in any appropriate manner.

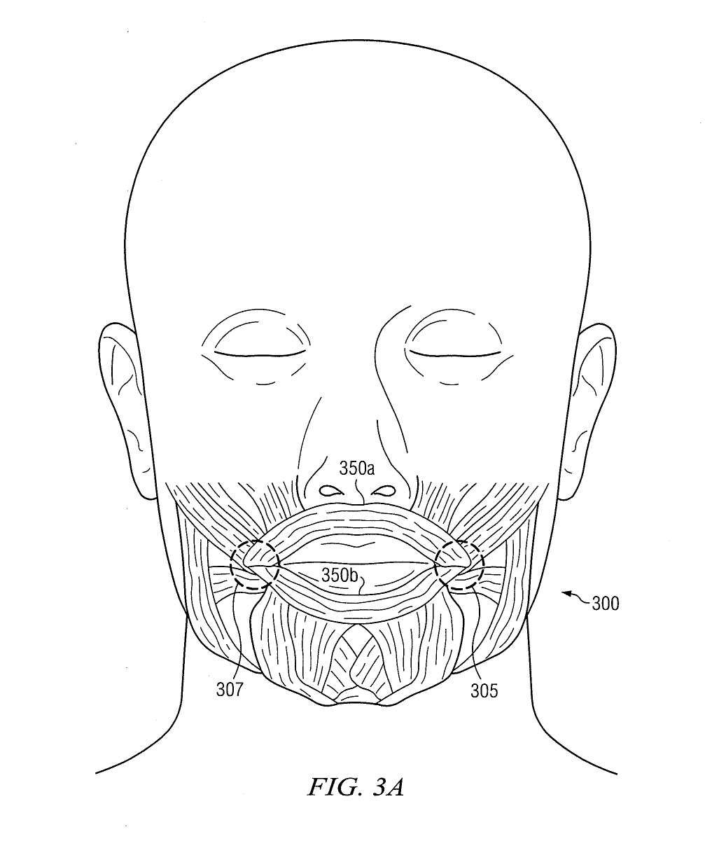

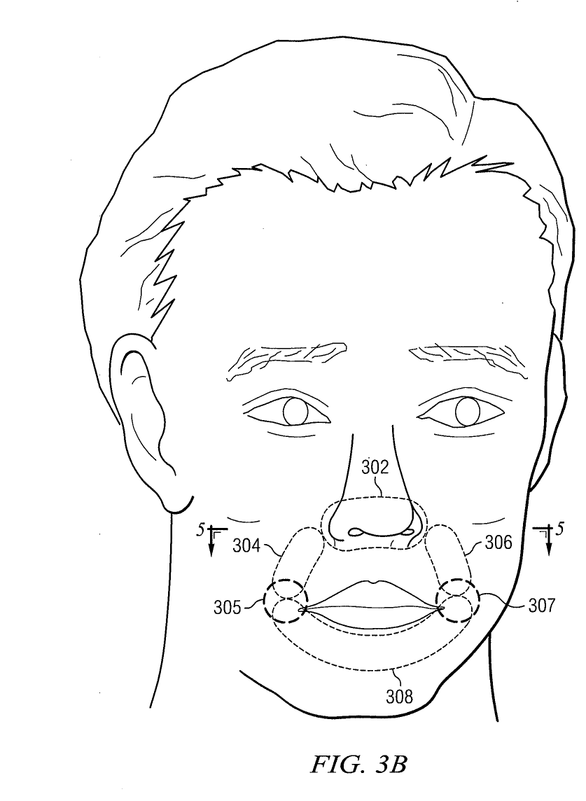

FIG. 3A illustrates an arrangement of muscle fibers 300 of a portion of particular user's face. In particular, the obicularis-oris muscle 350a and 350b is a sphincter muscle including numerous strata of muscular fibers surrounding the orifice of the mouth in different directions. Areas 305 and 307 generally indicate respective portions of the particular user's face proximate respective lateral perimeters of the obicularis-oris muscle 350. Area 305 is to the left of the user's mouth and area 307 is to the right of the user's mouth (from the user's perspective).

FIG. 3B illustrates a particular user's soft facial tissues that, when compressed by mask 102, corresponds to the shape of the custom-fitted sealing ridge 104 of mask 102. In this example, the particular user's soft facial tissues corresponding to sealing ridge 104 is generally indicated by multiple facial regions 304, 306, and 308. In certain embodiments, soft facial tissues corresponding to sealing ridge 104 may further include facial region 302, which includes soft cartilage tissues of the user's nose including portions surrounding the user's nostrils. Facial regions 304 and 306 indicate soft tissues of the user's face generally overlaying the user's nasal liable folds. In particular, facial region 304 extends from the left side of the user's nose to an area 305 proximate a lateral perimeter of the particular user's obicularis-oris muscle 350. Facial region 306 generally extends from the right side of the user's nose to another area 307 proximate a lateral perimeter of the particular user's obicularis-oris muscle 350. Facial region 308 indicates the lower extension of the particular user's obicularis-oris muscle 350b. For example, facial region 308 may extend from area 305 to the left of the user's mouth to area 307 to the right of the user's mouth (from the user's perspective).

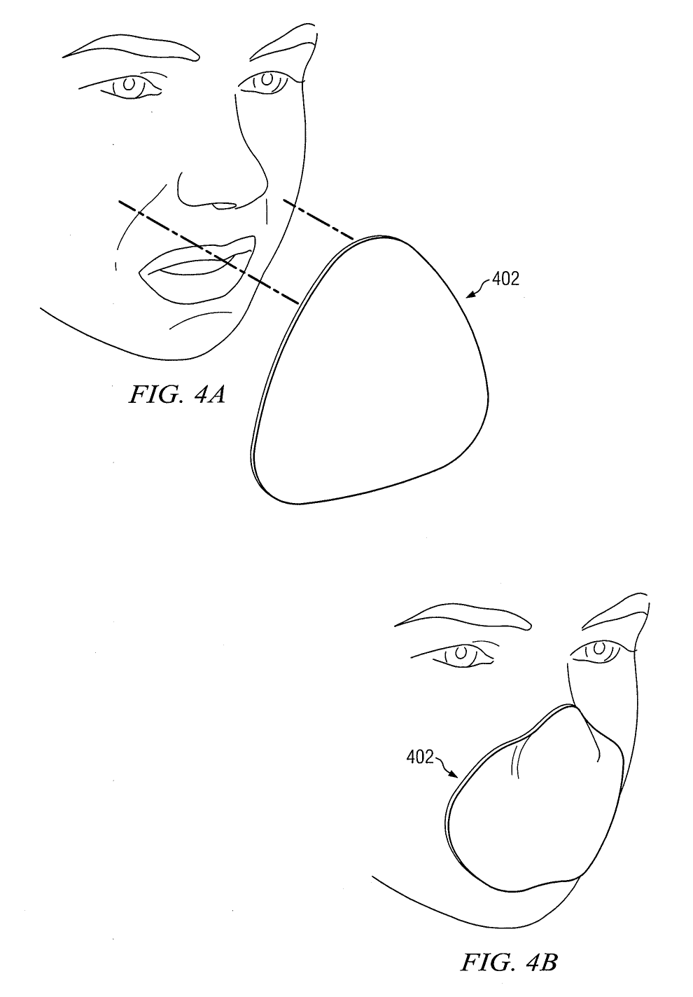

FIGS. 4A through 4C illustrate an example method of forming a custom-fitted mask 102 according to one embodiment. Referring to FIG. 4A, mask 102 begins as a deformable mask blank 402 made of a suitable deformable material. For example, mask blank 402 may include a suitable thermoplastic polymer and suitable fillers, stabilizers, coloring agents, antioxidants, antimicrobial agents, or other materials. As another example, mask blank 402 may include a light curing material such as the material sold under the name TRIAD by DENTSPLY INTERNATIONAL INC. Such materials are well known in various contexts to those skilled in the art.

In a more particular embodiment, mask blank 402 may include, possibly in addition to one or more other materials, one or more of the polycaprolactone polymers or other aliphatic polyesters that are described in U.S. Pat. Nos. 4,784,123 and 5,112,225 and product literature of UNION CARBIDE CORPORATION. One or more polycaprolactone polymers may have the formula:

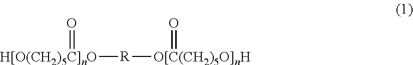

where R is an aliphatic hydrocarbon and n may range between approximately 300 to approximately 650. The TONE polycaprolactone polymers are described in U.S. Pat. Nos. 4,784,123 and 5,112,225 and product literature of UNION CARBIDE CORPORATION as including homopolymers, block copolymers, graft copolymers, or other polymers containing epsilon-caprolactone. Polymerization may be initiated using a diol, for example and without limitation, ethylene glycol, diethylene glycol, neopentyl glycol, butane diol, hexane diol, or any other appropriate diol. The diol may have the formula:

HO—R—OH (2)

where R is an aliphatic hydrocarbon. Where mask blank 402 includes one or more polycaprolactone polymers, any suitable polycaprolactone polymer or polymers may be used. In general, polycaprolactone polymers may display desirable dimensional stability and thermoplasticity during cooling, biocompatibility, and a variety of other characteristics making them suitable for use in forming mask blank 402.

As shown in FIG. 4A, mask blank 402 used in forming mask 102 may include a thin sheet of deformable material shaped to cover a portion of a generic user's face, including at least the portion of the generic user's nose surrounding the generic user's nostrils. For example, mask blank 402 may be sized and shaped such that it will cover only the soft cartilaginous portions of the generic user's nose and portions of the generic user's face to the sides of and below the soft cartilaginous portions of the generic user's nose, but may or may not cover the hard bony portions of the generic user's nose generally above the soft cartilaginous portions of the generic user's nose. In another embodiment, mask blank 402 may include a thin sheet of deformable material that is substantially flat except for a depression sufficient to accommodate a generic user's nose, including at least the portion of the generic user's nose surrounding the user's nostrils. Portions of such a mask blank 402 proximate the depression may be transitioned to a deformable state to adjust the fit of seal, thereby creating a custom-fitted mask. In another embodiment, mask blank 402 may include a thin sheet of deformable material that is substantially flat over its entire surface and does not include any depressions. Thin sheets of an appropriate deformable material may be obtained, for example, from CHESAPEAKE MEDICAL PRODUCTS, INC. In all cases, mask blank 402 may or may not include pre-formed holes positioned according to the position of a generic user's nostrils. Mask blank 402 may be formed using an injection molding process (i.e. deformable material is placed into an injection molding machine while in a liquid state), using a pressing process (i.e. deformable material is placed into a press while in a deformable state), or using any other suitable technique. Mask blank 402 may be sized for various classes of generic users, for example, large for men, medium for women, and small for children. In one embodiment, mask blank 402 may be mass produced quickly, inexpensively, and with high quality and uniformity. Mask blank 402 may include fittings 114, devices 116, or any other suitable features.

To form and fit mask 102 for a particular user using mask blank 402, mask blank 402 is placed in a deformable state. For example, where the deformable material of mask blank 402 includes one or more polycaprolactone polymers, mask blank 402 may be heated in a microwave oven, in water or other non-solvent neutral liquid, or in any other suitable manner to between approximately 140° F. and approximately 180° F. so as to place mask blank 402 in a deformable state. Before or after the deformable material solidifies into its final custom fitted shape, mask 102 is removed from the user's face. If desired, features such as fittings 114 and devices 116 may be coupled to mask blank 402 at this time if such features are not already coupled to mask blank 402. The deformable material may chemically bond to features such as fittings 114 and devices 116 while mask blank 402 is in a deformable state to produce an integral unit.

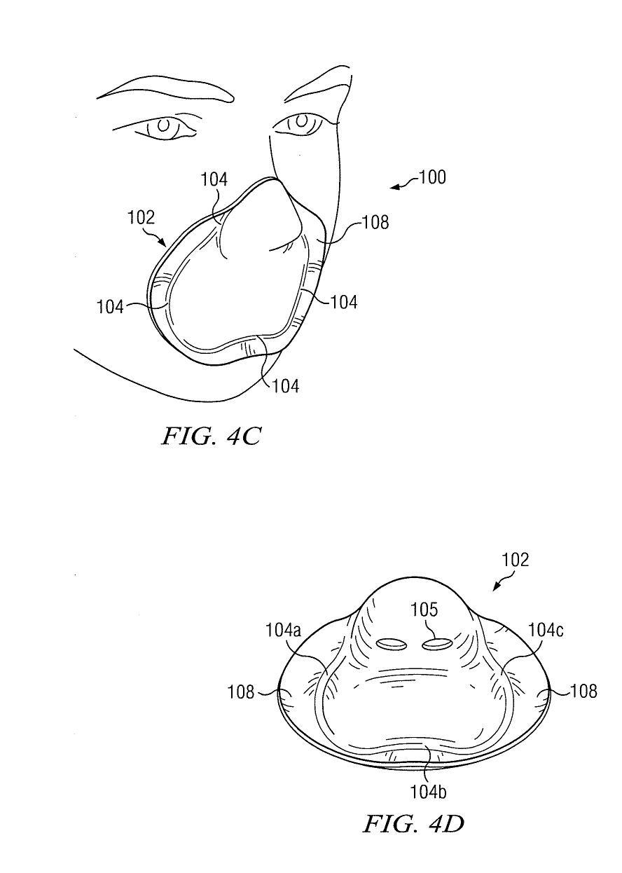

As shown in FIG. 4B, while mask blank 402 is in a deformable state it is brought in contact with the user's face. Mask blank 402 is then pressed against soft tissues of the user's face including, for example, one or more of regions 302, 304, 306, and/or 308 described previously with reference to FIG. 3B. In certain embodiments, the compressed shape of the soft facial tissues may be substantially different from the natural or uncompressed shape of the soft facial tissues, as explained further with reference to FIGS. 5A through 5D. The pressing of mask blank 402 against soft tissues of the particular user's face in particular regions forms a sealing ridge 104 corresponding to the compressed shape of the soft facial tissues in those particular regions, as shown in FIG. 4C.

Deformable material of mask blank 402 may cool and transition to a substantially non-deformable state to retain its custom-fitted shape including a substantially rigid, custom-fitted sealing ridge 104. Sealing ridge 104 may allow custom-fitted mask 102 to better conform to the unique shape of compressed facial tissues of the user's face and, as a result of the improved fit, to provide reduced leakage, increased comfort, and better performance.

FIG. 4D is a reversed view of mask 102 after the formation of substantially rigid, custom-fitted sealing ridge 104. The reversed view shows the inner surfaces of mask 102, including sealing ridge 104, configured to contact and form a custom-fit with the particular user's face. In this example, sealing ridge 104 includes three portions 104a, 104b, and 104c corresponding to compressed shapes of facial regions 304, 306, and 308, respectively, of the particular user's face. In particular, first portion 104a corresponds to a compressed shape of the user's face extending from the left side of the user's nose to area 305 proximate a lateral perimeter of the particular user's obicularis-oris muscle 350. Portion 104d corresponds to a compressed shape of the user's face extending from the right side of the user's nose to area 307 proximate a lateral perimeter of the particular user's obicularis-oris muscle 350. Portion 104b corresponds to a compressed shape of the user's face generally overlaying the lower extension of the particular user's obicularis-oris muscle 350b and generally extending from area 305 to area 307. In this example, portions 104a, 104b, and 104c form one continuous sealing ridge 104 extending from the left side of the user's nose, below the user's mouth, and to the right side of the user's nose. That is, portions 104a and 104b are joined together and portions 104b and 104c are joined together. Although portions 104a, 104b, and 104c form one continuous sealing ridge in this example, in alternative embodiments a gap or divot may separate one portion of sealing ridge 104 from the next closest portion of sealing ridge 104. Additionally, in alternative embodiments sealing ridge 104 may include a portion capable of forming a tight seal outwardly from soft cartilage of the user's nose.

In particular embodiments, mask 102 may include one or more holes 105 positioned proximate the user's nostrils, as shown in FIG. 4D. In certain embodiments, holes 105 may have been pre-formed features of mask blank 402. In alternative embodiments, holes 105 may be trimmed or otherwise formed after mask blank 402 is transitioned to a substantially non-deformable state.

FIGS. 5A through 5D illustrate various cross-sections of an example custom-fitted mask 102 at different stages of formation. In particular, FIGS. 5A, 5B, 5C, and 5D generally correspond to FIGS. 4A, 4B, 4C, and 4D, respectively, from the perspective of cross-sectional plane 5. As shown in FIG. 5B, the mask blank is transitioned to a substantially deformable state and allowed to conform to the natural or uncompressed shape of the particular the user's face. As shown in FIG. 5C, mask blank 402 is pressed against facial regions 304 and 308 of the particular user's face. The compression of these regions by mask blank 402 forms respective portions 104a and 104c of sealing ridge 104. Portions 104a and 104c of sealing ridge 104 are shaped in a manner corresponding to the compressed contours of facial regions 304 and 308, which differs from the natural or uncompressed contours of the same facial regions 304 and 308 illustrated in FIG. 5A. As shown in FIGS. 5C and 5D, a flange 108 portion is disposed exterior to and around sealing ridge 104. In the illustrated embodiment, the perimeter of flange 108 does not conform to the natural contour to of the particular user's face but rather is slightly separated from the user's face. Although the illustrated embodiment includes a flange 108 that does not conform to the natural contour of the particular user's face, alternative embodiments may include a flange that does conform to the particular user's face. Additionally, alternative embodiments may not include flange 108.

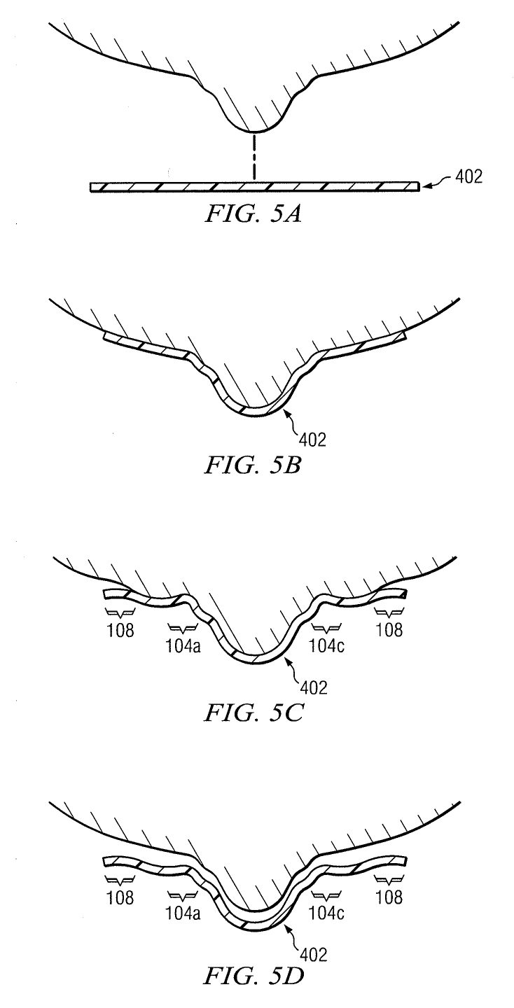

FIG. 6 illustrates an alternative mask 602 custom-fitted to another particular user according to one embodiment. In this example, mask 602 has a custom-fitted, substantially rigid sealing ridge 604. Sealing ridge 604 has a shape corresponding to compressed tissues of the particular user's face illustrated in FIG. 6. Sealing ridge 604 is shaped different from sealing ridge 104 at least because the user illustrated in FIG. 6 has facial features substantially different from the user illustrated in FIGS. 1 through 5C. As shown in FIG. 6, mask 602 covers the hard bony portions of the particular user's nose generally above the soft cartilaginous portions of the generic user's nose.

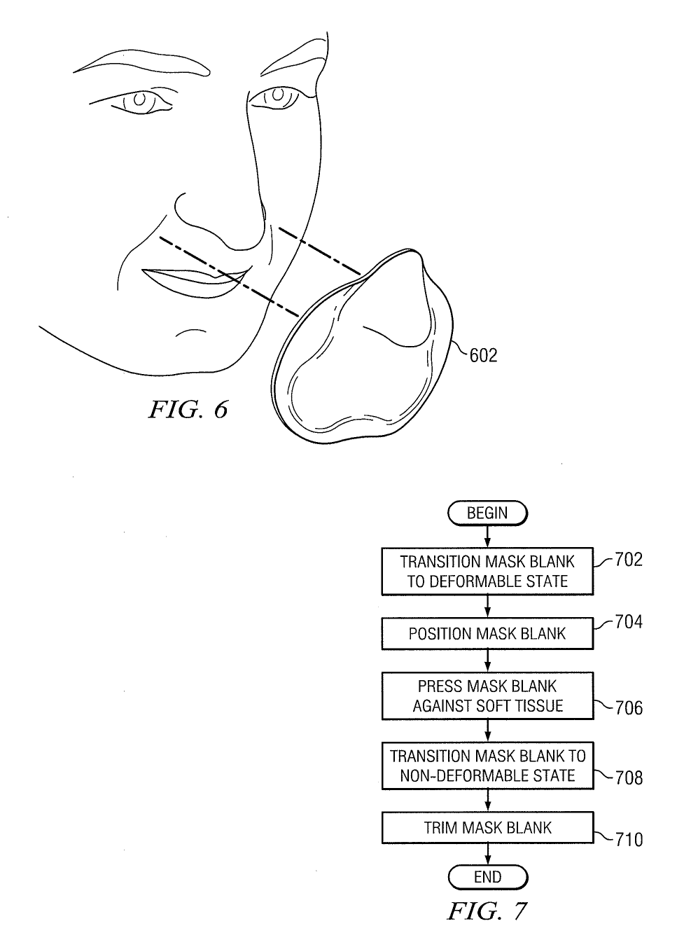

FIG. 7 illustrates a flowchart 700 of an example method for forming a custom-fitted mask for a particular user according to one embodiment. At step 702, mask blank 402 is transitioned into a substantially deformable state. For example, mask blank 402 may be transitioned into the substantially deformable state at least in part by exposing mask blank 402 to a temperature of approximately 140 degrees Fahrenheit or greater. At step 704, mask blank 402 is positioned to cover the particular user's mouth and at least a portion of the particular user's nose. In an alternative embodiment, selected portions of mask blank 402 may be transitioned to a substantially deformable state while other portions remain substantially non-deformable. For example, heated sand or water may be applied to various localized areas of mask blank 402 corresponding to sealing ridge 104.

At step 706, mask blank 402 is pressed against soft tissues of the particular user's face, such that the mask blank conforms to the compressed shape of the particular user's soft tissues. For example, mask blank 402 may be pressed against a first region of the particular user's face extending from the particular user's nose to a first area proximate a lateral perimeter of the particular user's obicularis-oris muscle 350. The first area may be located to the right of the particular user's mouth. In certain embodiments, mask blank 402 may be pressed against a second region of the particular user's face extending from the particular user's nose to a second area proximate a lateral perimeter of the particular user's obicularis-oris muscle 350. The second area may be located to the left of the particular user's mouth. In various embodiments, mask blank 402 may be pressed against a third region of the particular user's face extending from the first area to the second area. The third region may correspond to the lower extension of the particular user's obicularis-oris muscle 350b. In particular embodiments, the second and third regions of the particular user's face may each comprise soft tissues overlaying respective portions of the particular user's nasal-labial folds.

At step 708, mask blank 402 is allowed to transition to a substantially non-deformable state to form a substantially rigid, custom-fitted mask 102. For example, mask blank 402 may be transitioned to the substantially deformable state at least in part by exposing mask blank 402 to a temperature of approximately 100 degrees Fahrenheit or less. Mask 102 may include a substantially rigid custom-fitted sealing ridge 104 that has been custom shaped to correspond to the compressed shape of soft tissues of the particular user's face.

At step 710, the substantially rigid, custom-fitted mask 102 may be trimmed or otherwise reshaped. For example, rough or angular edges may be trimmed or smoothed for optimal fit and/or comfort. In addition, portions of mask 102 may be removed to form on or more holes 105 at positions corresponding to the user's mouth and/or nostrils. One or more buckles or other devices 116 may be attached to mask 102 to facilitate securing mask 102 to the particular user's face during use. In various embodiments, one or more fittings 114 may be attached to mask 102 to facilitate coupling mask 102 to an external system 110 and/or 210, such as a continuous positive air pressure (CPAP) system, a bi-level positive airway pressure (BiPAP) system, etc. The trimmed or otherwise reshaped mask 102 may be ready for use, for example, in treating sleep disordered breathing.

To assist in describing the features and interactions of certain components, relational terms have been used. For example, certain components have been described as being to the left or the right of particular features. It should be understood that these terms have been used to describe example implementations and are not intended to limit the scope of the claimed invention. To the contrary, in alternative embodiments, the spatial location of one or more of the components described may be reversed or altered.

The example methods disclosed herein may include more, fewer, or other steps. For example, in alternative embodiments mask blank 402 may be preformed with one or more holes 105, thereby minimizing or eliminating acts described above with reference to step 710. Additionally, steps may be performed in any suitable order. For example, portions of ridge 104 may be formed in any suitable order. Additionally, in alternative embodiments mask blank 402 may be trimmed before it is transitioned to a substantially non-deformable state. In particular embodiments, all or a portion of the steps disclosed herein may be wholly or partially performed by a user (e.g., a wearer of the oral appliance) and/or by someone else (e.g., physician, medical clinician, dentist, orthodontist, parent, etc.).

Although the masks disclosed herein are described primarily for use in treating breathing problems such as sleep disordered breathing, the present invention contemplates masks 102 and 602 being used or for any other suitable purpose for which a custom fitted mask may be used. As just one example, mask 102 and/or 602 may be used in delivering anesthetics during surgery or another medical procedure.

Although the present invention has been described in several embodiments, a myriad of changes, substitutions, variations, transformations, and modifications may be suggested to one skilled in the art, and it is intended that the present invention encompass such changes, substitutions, variations, transformations, and modifications as fall within the scope of the appended claims.

What is claimed is:

1. A method of forming a custom medical mask with a substantially rigid custom-fitted sealing ridge that has been custom shaped to correspond to a compressed shape of soft tissues of a particular user's face, the method comprising:
transitioning a mask blank to a substantially deformable state;
positioning the mask blank to cover the particular user's mouth and at least a portion of the particular user's nose;
pressing the mask blank against a first region of the particular user's face extending from the particular user's nose to a first area proximate a lateral perimeter of the particular user's obicularis-oris muscle, the first area located to the right of the particular user's mouth;
pressing the mask blank against a second region of the particular user's face extending from the particular user's nose to a second area proximate a lateral perimeter of the particular user's obicularis-oris muscle, the second area located to the left of the particular user's mouth; and
pressing the mask blank against a third region of the particular user's face extending from the first area to the second area, the third region corresponding to the lower extension of the particular user's obicularis-oris muscle;
wherein the substantially rigid custom-fitted sealing ridge conforms to the compressed shape of the particular user's soft tissues when the mask blank is pressed against the first, second, and third regions of the particular user's face.

2. The method of claim 1, further comprising allowing the mask blank to transition to a substantially non-deformable state, such that the mask blank transitioned to the substantially non-deformable state forms the substantially rigid custom-fitted sealing ridge that has been custom shaped to correspond to the compressed shape of soft tissues of the particular user's face.

3. The method of claim 1, wherein the first and second regions of the particular user's face each comprise respective portions of the particular user's nasal-labial folds.

4. The method of claim 1, further comprising pressing the mask blank against a fourth region of the particular user's face corresponding to a soft cartilaginous portion of the particular user's nose.

5. The method of claim 1, wherein the substantially rigid custom-fitted sealing ridge comprises first, second, and third ridge areas corresponding to the first, second, and third regions of the particular user's face, respectively; and
wherein the third ridge area is joined to the first ridge area and to the second ridge area.

6. The method of claim 1, wherein the mask blank comprises a thermoplastic polymer.

7. The method of claim 4, wherein the thermoplastic polymer comprises a polycaprolactone polymer.

8. The method of claim 1, further comprising trimming portions of the mask blank transitioned to the substantially non-deformable state, the trimming comprising forming at least one hole corresponding to a nostril of the particular user.

9. The method of claim 1, wherein the substantially rigid custom-fitted sealing ridge has a maximum width within the range of approximately 2 millimeters to 2 centimeters, the maximum width being less than, and transverse to the direction of, a length of the sealing ridge.

10. The method of claim 1, wherein the mask blank transitioned to the substantially non-deformable state comprises a flange disposed exterior to an internal sealing region defined at least in part by the substantially rigid custom-fitted sealing ridge.

11. A custom medical mask with a substantially rigid custom-fitted sealing ridge that has been custom shaped to correspond to a compressed shape of soft tissues of a particular user's face, the custom medical mask formed by a process comprising:
transitioning a mask blank to a substantially deformable state;
positioning the mask blank to cover the particular user's mouth and at least a portion of the particular user's nose;
pressing the mask blank against a first region of the particular user's face extending from the particular user's nose to a first area proximate a lateral perimeter of the particular user's obicularis-oris muscle, the first area located to the right of the particular user's mouth;
pressing the mask blank against a second region of the particular user's face extending from the particular user's nose to a second area proximate a lateral perimeter of the particular user's obicularis-oris muscle, the second area located to the left of the particular user's mouth; and
pressing the mask blank against a third region of the particular user's face extending from the first area to the second area, the third region corresponding to the lower extension of the particular user's obicularis-oris muscle;
wherein the a substantially rigid custom-fitted sealing ridge conforms to the compressed shape of the particular user's soft tissues when the mask blank is pressed against the first, second, and third regions of the particular user's face.

12. The custom medical mask of claim 11, wherein the process further comprises allowing the mask blank to transition to a substantially non-deformable state, such that the mask blank transitioned to the substantially non-deformable state forms the substantially rigid custom-fitted sealing ridge that has been custom shaped to correspond to the compressed shape of soft tissues of the particular user's face.

13. The custom medical mask of claim 11, wherein the first and second regions of the particular user's face each comprise respective portions of the particular user's nasal-labial folds.

14. The custom medical mask of claim 11, wherein the process further comprises pressing the mask blank against a fourth region of the particular user's face corresponding to a soft cartilaginous portion of the particular user's nose.

15. The custom medical mask of claim 11, wherein the substantially rigid custom-fitted sealing ridge comprises first, second, and third ridge areas corresponding to the first, second, and third regions of the particular user's face, respectively; and
wherein the third ridge area is joined to the first ridge area and to the second ridge area.

16. The custom medical mask of claim 11, wherein the mask blank comprises a thermoplastic polymer.

17. The custom medical mask of claim 16, wherein the thermoplastic polymer comprises a polycaprolactone polymer.

18. The custom medical mask of claim 11, the process further comprises trimming portions of the mask blank transitioned to the substantially non-deformable state, the trimming comprising forming at least one hole corresponding to a nostril of the particular user.

19. The custom medical mask of claim 11, wherein the substantially rigid custom-fitted sealing ridge has a maximum width within the range of approximately 2 millimeters to 2 centimeters, the maximum width being less than, and transverse to the direction of, a length of the sealing ridge.

20. The custom medical mask of claim 11, wherein the mask blank transitioned to the substantially non-deformable state comprises a flange disposed exterior to an internal sealing region defined at least in part by the substantially rigid custom-fitted sealing ridge.

21. A custom medical mask comprising:
a substantially rigid custom-fitted sealing ridge that has been custom shaped to correspond to a compressed shape of soft tissues of a particular user's face, the substantially rigid custom-fitted sealing ridge comprising:
a first ridge corresponding to a first portion of the particular user's face extending from the particular user's nose to a first area proximate a lateral perimeter of the particular user's obicularis-oris muscle, the first area located to the right of the particular user's mouth;
a second ridge corresponding to a second portion of the particular user's face extending from the particular user's nose to a second area proximate a lateral perimeter of the particular user's obicularis-oris muscle, the second area located to the left of the particular user's mouth; and
a third ridge corresponding to a third portion of the particular user's face extending from the first area to the second area, the third portion corresponding to the lower extension of the particular user's obicularis-oris muscle;
wherein the substantially rigid custom-fitted sealing ridge is configured to form a custom-fit seal by conforming to the compressed shape of the particular user's soft tissues when the custom medical mask is pressed against the first, second, and third portions of the particular user's face.

22. The custom medical mask of claim 21, wherein the first and second regions of the particular user's face each comprise respective portions of the particular user's nasal-labial folds.

23. The custom medical mask of claim 21, wherein the substantially rigid custom-fitted sealing ridge further comprises a fourth ridge corresponding to a soft cartilaginous portion of the particular user's nose.

24. The custom medical mask of claim 21, wherein the third ridge is joined to the first ridge and to the second ridge.

25. The custom medical mask of claim 21, wherein the custom medical mask comprises a thermoplastic polymer.

26. The custom medical mask of claim 25, wherein the custom medical mask comprises a polycaprolactone polymer.

27. The custom medical mask of claim 21, wherein the custom medical mask comprises at least one hole corresponding to a nostril of the particular user.

28. The custom medical mask of claim 21, wherein the custom medical mask comprises a flange disposed exterior to an internal sealing region defined at least in part by the substantially rigid custom-fitted sealing ridge.

29. The custom medical mask of claim 21, wherein the substantially rigid custom-fitted sealing ridge has a maximum width within the range of approximately 2 millimeters to 2 centimeters, the maximum width being less than, and transverse to the direction of, a length of the sealing ridge.

30. A custom medical mask comprising:
a substantially rigid custom-fitted sealing ridge that has been custom shaped to correspond to a compressed shape of soft tissues of a particular user's face, the substantially rigid custom-fitted sealing ridge comprising:
a first ridge corresponding to a first portion of the particular user's face extending from the particular user's nose to a first area proximate a lateral perimeter of the particular user's obicularis-oris muscle, the first area located to the right of the particular user's mouth;
a second ridge corresponding to a second portion of the particular user's face extending from the particular user's nose to a second area proximate a lateral perimeter of the particular user's obicularis-oris muscle, the second area located to the left of the particular user's mouth;
a third ridge corresponding to a third portion of the particular user's face extending from the first area to the second area, the third portion corresponding to the lower extension of the particular user's obicularis-oris muscle; and
a fourth ridge corresponding to a soft cartilaginous portion of the particular user's nose;
wherein the substantially rigid custom-fitted sealing ridge is configured to form a custom-fit seal by conforming to the compressed shape of the particular user's soft tissues when the custom medical mask is pressed against the first, second, and third portions of the particular user's face;
wherein the first and second portions of the particular user's face each comprise soft tissue overlaying respective portions of the particular user's nasal-labial folds;
wherein the third ridge is joined to the first ridge and to the second ridge; and
wherein the custom medical mask comprises a flange disposed exterior to an internal sealing region defined at least in part by the substantially rigid custom-fitted sealing ridge.

31. The custom medical mask of claim 21, wherein the custom medical mask comprises a thermoplastic polymer.

32. The custom medical mask of claim 24, wherein the custom medical mask comprises a polycaprolactone polymer.
